(19) United States
(12) Patent Application Publication
(10) Pub. No.: US 2022/0323344 A1
(43) Pub. Date: Oct. 13, 2022

(54) TRANSDERMAL PENETRATION BY MODULATING EPITHELIAL JUNCTIONS

(71) Applicant: Dyve Biosciences, Inc., Camarillo, CA (US)

(72) Inventors: Nathan FITZSIMMONS, Newbury Park, CA (US); Ryan BEAL, Camarillo, CA (US); Brandon SAND, Camarillo, CA (US)

(21) Appl. No.: 17/830,236

(22) Filed: Jun. 1, 2022

Related U.S. Application Data
(63) Continuation of application No. PCT/US2020/062953, filed on Dec. 2, 2020.
(60) Provisional application No. 62/942,465, filed on Dec. 2, 2019.

(51) Int. Cl. A61K 9/00; A61K 47/12

(52) U.S. Cl. CPC A61K 9/0014; A61K 47/12

(57) ABSTRACT

Embodiments include a transdermal delivery formulation and method for transdermal delivery of an active agent for systemic distribution. A formulation can be applied to skin, nail or hair follicle of a subject. After penetrating the stratum corneum, the agent can pass through other layers of skin as junctional proteins and/or acto-myosin belts between cells are modulated. The formulation can include one or more agents to treat a disease, ailment or an anesthetic to alleviate pain. Alternatively, it can include one or more nutrients, vitamins, minerals or supplements to promote health and well-being.

### CROSS REFERENCE TO RELATED APPLICATIONS

This application is a Continuation Application of International Application No. PCT/US2020/062953, filed Dec. 2, 2020, which claims the benefit of U.S. Provisional Application No. 62/942,465, filed Dec. 2, 2019, all of which are hereby incorporated by reference in their entirety.

### FIELD OF THE INVENTION

The invention relates generally to topical administration of medicaments. More specifically, it relates to modulating epithelial junctions and the actin cytoskeleton to increase absorption of medicaments through the skin of a subject.

### BACKGROUND

Topical administration describes the application of a substance to a surface of the skin. The term is often used to describe the application of a cream, foam, gel, lotion or ointment to the skin or mucous membranes. The high keratinization of skin cells and their dense packing creates, in most cases, a barrier impermeable to penetration. Because of this, most substances are not absorbed through the skin.

The term transdermal administration refers to applying a substance onto the skin so that it is absorbed into the body for systemic distribution. Transdermal administration can be effective for hydrophobic chemicals such as steroid hormones. For example, transdermal patches are a common means of administering steroidal drugs for birth control, hormone replacement therapy and prevention of motion sickness.

Medicaments that are not hydrophobic chemicals are typically unsuited for topical administration. To be effective, the active drug or agent in a topical composition must penetrate the skin, which is structurally complex and relatively thick. Molecules moving through the skin must first penetrate the stratum corneum and any material on its surface. The molecules must then penetrate the epidermis, the papillary dermis, and the capillary walls into the vascular system or lymphatic system. To be absorbed, the molecules must overcome a different resistance to penetration in each layer.

Strategies have been devised to improve transdermal administration of medicaments. These strategies can be categorized as either physical, chemical, mechanical or biochemical. Combinations of these strategies can also increase efficacy or extend the time for transdermal delivery. Physical techniques include abrasion and tape stripping, which physically break open the skin. Another physical method is prolonged occlusion, which alters the barrier properties of the stratum corneum. After 24 to 28 hours of occlusion with resultant hydration, corneocytes swell, intercellular spaces become distended, and the lacunar network becomes dilated. Distention of the lacunae eventually leads to connections with an otherwise discontinuous system. This creates pores in the stratum corneum interstices through which polar and non-polar substances can penetrate more easily.

Other approaches include the use of Chemical Permeation Enhancers. Chemical Permeation Enhancers (CPEs) are molecules that interact with the constituents of skin's outermost layer, the stratum corneum (SC), and increase its permeability. However, despite efforts at improving them, CPEs are minimally effective in increasing the rate at which drugs permeate the skin. CPEs can also cause skin damage, irritation and sensitization. Further, they are generally ineffective with high molecular weight drugs such as peptides, proteins and nucleic acids.

Although a variety of methods can be used to enhance transdermal drug delivery, these methods have limitations. Most efforts to enhance transdermal penetration have focused on the outermost layer of the skin, the stratum corneum. They typically rely on harsh solvents (e.g., alcohols, DMSO) or patch-based systems. This approach limits the molecular size, lipophilicity, and potency of drugs that can be used. In essence, current approaches are largely limited to small, lipophilic, and highly potent drugs.

An improved method of transdermal penetration should overcome the barrier presented by the stratum corneum as well as the deeper layers of skin. Further, it should do so without harsh solvents and work with high molecular weight agents such as peptides, proteins and nucleic acids

### SUMMARY OF THE INVENTION

Aspects of the present invention fulfill these needs and provide further related advantages as described in the following summary.

The present disclosure solves the problems described above by providing therapeutic agent formulations with improved penetration of agents administered transdermally.

In one aspect, disclosed herein is a transdermal delivery formulation, wherein the transdermal delivery formulation includes one or more junctional protein modulators. The formulation can also include an active agent to treat a disease such as a cancer, a kidney disease, gout, melasma, a heart condition or a dermal disease.

In another aspect, the transdermal delivery formulation includes one or more junctional protein modulators such as Clostridium perfringens enterotoxin, zona occludens toxin (ZOT), AT1002, chitosan, a calcium chelator, sodium caprate, FDFWITP, PN159, 1-1-palmitoyl-2-glutaroyl-sn-glycero-3-phosphocholine (PGPC), oleic acid or histamine. The transdermal delivery formulation can also include one or more acto-myosin belt modulators such as a calcium chelator.

In another aspect, disclosed herein is a method of transdermal delivery of an active agent, comprising steps of (a) applying a transdermal delivery formulation to skin, nail or hair follicle of a subject, (b) penetrating the stratum corneum, (c) modulating one or more junctional proteins and (d) modulating acto-myosin belts between cells.

In another aspect, disclosed herein is a method of enhancing absorption of an agent across epithelial cells of the intestine comprising a step of modulating one or more junctional proteins. In another aspect, disclosed herein is a method of enhancing absorption of an agent through the blood brain barrier comprising a step of modulating one or more junctional proteins.

Other features and advantages of aspects of the present invention will become apparent from the following more detailed description, taken in conjunction with the accompanying drawings, which illustrate, by way of example, the principles of aspects of the invention.

### Definitions

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can in certain instances be used interchangeably.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

As applicable, the terms “about” or “generally”, as used herein in the specification and appended claims, and unless otherwise indicated, means a margin of +/−20%. Also, as applicable, the term “substantially” as used herein in the specification and appended claims, unless otherwise indicated, means a margin of +/−10%. It is to be appreciated that not all uses of the above terms are quantifiable such that the referenced ranges can be applied.

The term “subject” or “patient” refers to any single animal, more preferably a mammal (including such non-human animals as, for example, dogs, cats, horses, rabbits, zoo animals, cows, pigs, sheep, and non-human primates) for which treatment is desired. Most preferably, the patient herein is a human.

The term “actin cytoskeleton” or “actin skeleton” refers to a complex network of polarized filaments that is involved in many essential processes including motility and cytokinesis, tumor cell transformation and metastasis.

The term “actomyosin” refers to the actin-myosin complex that forms within the cytoskeleton. Actomyosin is inherently contractile as the myosin motor protein can pull on actin filaments. This property gives rise to contractile fibers that enable cell motility and force generation at the sub-cellular level.

The term “acto-myosin belt,” “perijunctional actomyosin ring” or “PAMR” refers to a circumferential actomyosin belt, which is composed of F-actin-myosin II bundles located along apical cell-cell junctions. In epithelial cells, myosin-II-dependent forces regulate many aspects of animal morphogenesis, such as apical constriction, cell intercalation, cell sorting, and the formation and maintenance of the adherens junction.

The term “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An active agent can be a primary active agent, or in other words, the component(s) of a composition to which the whole or part of the effect of the composition is attributed. An active agent can be a secondary agent, or in other words, the component(s) of a composition to which an additional part and/or other effect of the composition is attributed.

The term “anesthetic” refers to a substance that induces insensitivity to pain. While there are many drugs that can be used intravenously to produce anesthesia or sedation, the most common are barbiturates, amobarbital (Amytal), methohexital (Brevital), thiamylal (Surital), benzodiazepines, diazepam, lorazepam, midazolam, etomidate, ketamine and propofol.

The term “AT-1002” refers to the active domain of Vibrio cholerae's second toxin, zonula occludens toxin (ZOT). AT-1002, a hexamer peptide, has been shown to cause the redistribution of ZO-1 away from cell junctions. AT-1002 can also activate src and mitogen activated protein (MAP) kinase pathways, increased ZO-1 tyrosine phosphorylation, and rearrangement of actin filaments.

The term “chitosan” refers to an amino polysaccharide, produced from the deacetylation of chitin obtained from crustaceans and insects.

The term “Clostridium perfringens enterotoxin” refers to toxins produced by Clostridium species. Clostridial species are one of the major causes of food poisoning/gastrointestinal illnesses. The protein can destroy the cell membrane structure of animals by binding to claudin family proteins. These are components of tight junctions of the epithelial cell membrane.

The term “FDFWITP” refers to a peptide that has been recognized as a potential tight junction modulator.

The term “histamine” refers an organic nitrogenous compound involved in local immune responses, as well as regulating physiological function in the gut and acting as a neurotransmitter for the brain, spinal cord, and uterus. Structurally, it is an imidazole ring attached to an ethylamine chain. Under physiological conditions, the amino group of the side-chain is protonated.

The term “lipid raft” refers to areas within the lipid membrane of a cell. The plasma membranes of cells contain combinations of glycosphingolipids, cholesterol and protein receptors organized in glycolipoprotein lipid microdomains termed “lipid rafts.” Lipid rafts can influence membrane fluidity and membrane protein trafficking.

The term “junctional proteins” or “junctional complex” refers to anchoring proteins that extend through the plasma membrane to link cytoskeletal proteins in one cell to cytoskeletal proteins in neighboring cells as well as to proteins in the extracellular matrix.

The term “junctional protein modulator” refers to a chemical or agent that alters the structure or function of junctional proteins.

The term “tight junction,” “occluding junction” or “zonulae occludens” refers to a multiprotein junctional complex whose general function is to prevent leakage of transported solutes and water and seals the paracellular pathway. Tight junctions can also serve as leaky pathways by forming selective channels for small cations, anions, or water.

The term “zonula occludens toxin” or “ZOT” is produced by Vibrio cholerae and has the ability to increase mucosal permeability by reversibly affecting the structure of tight junctions.

All numerical designations, e.g., pH, temperature, time, concentration, and molecular weight, including ranges, are to be understood as approximations in accordance with common practice in the art. When used herein, the term “about” may connote variation (+) or (−) 1%, 5% or 10% of the stated amount, as appropriate given the context. It is to be understood, although not always explicitly stated, that the reagents described herein are merely exemplary and that equivalents of such are known in the art.

Many known and useful compounds and the like can be found in Remington's Pharmaceutical Sciences (13^{th} Ed), Mack Publishing Company, Easton, Pa.—a standard reference for various types of administration. As used herein, the term “formulation(s)” means a combination of at least one active ingredient with one or more other ingredient, also commonly referred to as excipients, which may be independently active or inactive. The term “formulation” may or may not refer to a pharmaceutically acceptable composition for administration to humans or animals and may include compositions that are useful intermediates for storage or research purposes.

In an embodiment, a “pharmaceutical composition” is intended to include the combination of an active agent with a carrier, inert or active, in a sterile composition suitable for diagnostic or therapeutic use in vitro, in vivo or ex vivo. In one aspect, the pharmaceutical composition is substantially free of endotoxins or is non-toxic to recipients at the dosage or concentration employed.

In an embodiment, “an effective amount” refers to the amount of the defined component sufficient to achieve the desired chemical composition or the desired biological and/or therapeutic result. In an embodiment, that result can be the desired pH or chemical or biological characteristic, e.g., stability of the formulation. In other embodiments, the desired result is the alleviation or amelioration of the signs, symptoms, or causes of a disease, or any other desired alteration of a biological system. When the desired result is a therapeutic response, the effective amount will vary depending upon the specific disease or symptom to be treated or alleviated, the age, gender and weight of the subject to be treated, the dosing regimen of the formulation, the severity of the disease condition, the manner of administration and the like, all of which can be determined readily by one of skill in the art. A desired effect may, without necessarily being therapeutic, also be a cosmetic effect, in particular for treatment for disorders of the skin described herein.

In an embodiment, a “subject” of diagnosis or treatment is a prokaryotic or a eukaryotic cell, a tissue culture, a tissue or an animal, e.g. a mammal, including a human. Non-human animals subject to diagnosis or treatment include, for example a simian, a murine, a canine, a leporid, such as a rabbit, livestock, sport animals, and pets.

In an embodiment, as used herein, the terms “treating,” “treatment” and the like are used herein to mean obtaining a desired pharmacologic and/or physiologic effect. The effect may be prophylactic in terms of completely or partially preventing a disorder or sign or symptom thereof, and/or may be therapeutic in terms of amelioration of the symptoms of the disease or infection, or a partial or complete cure for a disorder and/or adverse effect attributable to the disorder.

For purposes herein, a formulation, a formulation for transdermal delivery and a transdermal delivery formulation are each a formulation for transdermal delivery, including, the transdermal delivery of an active ingredient for the treatment of a syndrome and or a disease in an individual.

### DESCRIPTION OF EMBODIMENTS OF THE INVENTION

Embodiments include a novel formulations and methods of penetrating the stratum corneum by using tight junction modulators. Embodiments also include formulations for transdermal administration of one or more active agents. The transdermal delivery formulation can include one or more junctional protein modulators. The modulators can signal protein kinases to modify or open tight junctions. The transdermal deliver formulation can also include one or more agents that modulate the acto-myosin belt between cells. The formulation can be used to transdermally administer a range of active agents. Active agents can be, for example, medicaments to treat a disease such as a cancer, a kidney disease, gout, melasma, a heart condition or a dermal disease. Active agents can also be nutrients, vitamins, minerals or supplements to promote health and well-being.

### Transdermal Delivery Formulation Components

Cellular junctions, tight junctions and adherens junctions serve a structural role between skin cells. In addition however, it is also clear that they can form additional “filters” or “sieves” when it comes to larger molecules. The implications for transdermal penetration were inspired by epithelial penetration/integrity studies pertaining to normal physiologic function in other epithelial cell barrier regions, namely the intestinal tract, nasal passages, and respiratory tract. Research has been conducted on the barrier function for proper gastrointestinal function, pharmaceutical absorption, and water retention/elimination. In addition, oncology research explored gap and tight junction function as it relates to tumor invasion and metastases and the pathogenesis of some bacteria and viruses (e.g., hepatitis virus C, Clostridium perfringens enterotoxin (CPE), retroviruses, coxsackie, and adenoviruses) and inflammatory bowel disease (IBD)). The invention includes an approach to leverage the principles identified in other epithelial systems to a robust transdermal penetration system for macromolecules.

Absorption across epithelial cells has been studied extensively in relation to digestion. Absorption occurs across cells of the digestive system as nutrients are passed to the circulatory and lymphatic capillaries through osmosis, active transport and diffusion. The intestinal walls are lined with epithelial cells that have numerous microvilli to improve the absorption of nutrients by increasing the surface area of the intestine. However, not all drugs are readily absorbed through the intestine. For this reason, junctional proteins have been studied for their potential in enhancing drug absorption.

The epithelial and endothelial barriers of the human body can be considered obstacles for drug delivery to the systemic circulation and to organs with unique environment and homeostasis, like the central nervous system. Several transport routes exist in these barriers, which potentially can be exploited for enhancing drug permeability. While lipophilic molecules can diffuse across the cellular plasma membranes, the junctional complexes restrict or completely block the free passage of hydrophilic molecules through the paracellular clefts. Absorption or permeability enhancers developed in recent years for modifying intercellular junctions and paracellular permeability have unspecific modes of action. Recent advances have led to the discovery of an increasing number of integral membrane, adaptor, regulator and signaling proteins in tight and adherens junctions. Tight junction modulators can directly target tight or adherens junction proteins, the signaling pathways regulating junctional function, or tight junction associated lipid raft microdomains.

Modulators acting directly on tight junctions include peptides derived from zonula occludens toxin, or Clostridium perfringens enterotoxin, peptides selected by phage display that bind to integral membrane tight junction proteins, and lipid modulators. They can reversibly increase paracellular transport and drug delivery with less toxicity than previous absorption enhancers and have potential use as pharmaceutical excipients to improve drug delivery across epithelial barriers and the blood-brain barrier.

Several tight junction (TJ) proteins have been identified in humans as well as porcine, murine and canine skin. In addition to the interfollicular epidermis, TJ proteins are also found in hair follicles and in sweat glands with diverse localization patterns but also colocalizing at the areas where the outermost living layers face the outside. Alterations in TJ protein expression and localization have been observed in skin diseases, including neonatal ichthyosis sclerosing cholangitis (NISCH) syndrome (Cldn-1 knockout), atopic dermatitis, ichthyosis vulgaris and psoriasis.

Additionally, there is thick band of actin cytoskeleton proximal to the junctional proteins that is referred to as the perijunctional actomyosin ring (PAMR) or acto-myosin belt. This acto-myosin belt similarly serves a structural role, but when specifically activated it can serve as a physical disruption of paracellular barrier function. An individual cell can be pulled away from another adjacent cell by squeezing/contracting it via the actomyosin ring. Gaps between cells will invariably form when groups of adjacent cells are contracted.

The lack of transdermal focus on these structures is not surprising given that the primary barrier for skin penetration is the stratum corneum and existing technologies have been only modestly successful at penetrating this barrier leaving the deeper structures out of reach. That said, by leveraging newer penetration technologies it is possible to deliver junctional protein modulating agents in the skin. This leads to increases in the speed and efficiency of skin penetration and allows the penetration of molecules of much larger size, varying lipophilicity, and with lower potency (i.e., in higher doses) than previously possible.

### Junctional Protein Modulators

Several agents have been shown to affect the structure and activity of tight junction (TJ) proteins. Their mechanism of action may involve one or more of protein kinase A, C and G (PKA, PKC, PKG), Rho kinases, myosin light chain kinase (MLCK), and/or the mitogen activated protein kinase (MAPK) system.

Junctional protein modulators can include Clostridium perfringens enterotoxin (cCPE), ZOT as well as AT1002 (a ZOT-derived peptide), chitosan, (a cationic polymer) can increase TJ permeability via alteration of intracellular pH. Calcium chelators (e.g. EGTA, EDTA and BAPTA) can open TJs and adherens junctions via the activation of PKC. Sodium caprate (C10) can lead to a redistribution of the cytoskeleton and TJ proteins ZO-1 and occludin via phospholipase C-dependent inositol triphosphate/diacylglycerol pathways. Peptides (FDFWITP, PN159) can decrease TJ barrier function in nasal epithelia. Several lipids (e.g. 1-1-palmitoyl-2-glutaroyl-sn-glycero-3-phosphocholine (PGPC) or oleic acid) have been shown to reduce paracellular barrier function (e.g. in gastrointestinal, alveolar and blood-brain barriers).

Other junctional protein modulators can include bile acids, sphingolipid sphingosine-1-phosphate (S1P), Dihydro-S1P, prostanoids (e.g., prostaglandins), leukotrienes, arachidonic acid, eicosapentaenoic acid, ergot alkaloids (e.g. ergotamine, ergonovine, dihydroergotamine), calyculin-A, okadaic acid and anticholinesterase drugs (e.g., neostigmine, pyridostigmine, cholinergic drug, eserine, galantamine, donepezil, rivastigmine).

Histamine can also alter tight junction proteins. In recent studies, the histamine effect was observed in a dose range of 0.1— 100 microM. In addition, the histamine receptor H1R agonist, 2-pyridylethylamine, suppressed keratinocyte differentiation to the same extent as did histamine and thrombin. Further, ethanol and acetaldehyde can cause a rapid and synergistic elevation of intracellular calcium. Ethanol and acetaldehyde can synergistically disrupt tight junctions by a mechanism involving calcium, oxidative stress, Src kinase and MLCK.

The mechanism of action of modulators can involve protein kinase C (PKC) and an elevation of intracellular Ca2+ levels by phorbol esters or Ca2+ ionophore A23187. Ca2+ chelators can also activate PKC. Pathological stimuli, like oxidative stress, cytokines (tumour necrosis factor-α (TNF-α), etc.), and vascular endothelial growth factor (VEGF), as well as toxins from infectious agents, HIV-1 gp120, Vibrio cholerae zonula occludens toxin (Zot), Clostridium difficile toxin A, Escherichia coli OmpA all use PKC signaling to open tight junctions. Bryostatin 1, a novel anticancer agent and a non-phorbol ester stimulator of PKC transiently increases TJ permeability in epithelial cells through rapid downregulation of PKC-α.

Protein kinase A (PKA) can decrease intracellular cyclic AMP by phosphodiesterase 4 activators, or inhibition of peptide hormone adrenomedullin. Protein kinase G (PKG) can act via soluble guanylate cyclase activation and elevation of cyclic GMP. The bradykinin B2 receptor agonist Cereport can act through this pathway to reversibly open brain endothelial TJs. Pathological conditions, like hypoxia, ischemia or excessive nitric oxide (NO) release, either endogenous, or from NO-donors, also activate soluble guanylate cyclase and PKG and increase blood brain barrier (BBB) permeability.

Myosin light chain kinase (MLCK) activators have also been studied as modulators of TJ proteins. One of the main effector mechanisms in TJ regulation is the phosphorylation of myosin light chain (MLC) by MLCK leading to the contraction of the acto-myosin belt and disassembly of TJs. Cytokines, bacterial or viral pathogens, removal of extracellular Ca2+, as well as bile acids, used as absorption enhancers (AEs), trigger TJ opening via MLCK activation. The vasoactive mediator histamine and the lysophosphatidic acid (LPA) can also compromise the integrity of endothelial cells by MLCK activation. Further, MAPK can modulate paracellular permeability of TJs by regulating the expression of several TJ proteins. Ethanol, proinflammatory cytokines, bile salts, reactive oxygen species (ROS), pathogenic factors of viruses and bacteria can have a similar effect.

Rho kinases have also been studied as modulators of TJ proteins. Activation of RhoA, Rac1 and Cdc42, members of the Rho family of GTPases can disrupt epithelial tight junctions. RhoA interacts with the PKC and MLCK pathways and regulates TJ disassembly through phosphorylation of integral membrane TJ proteins occludin and claudin-5 and scaffolding proteins ZO-1 and ZO-2. The level of RhoA is critical for TJ regulation, and either inactivation of it by C3 transferase, a specific inhibitor, or activation by cytokine interferon-γ and 2-methoxyestradiol, a microtubule destabilizing drug, leads to TJ disassembly. RhoA participates in reduced brain endothelial TJ functions after LPA treatment, and in ROS-induced alterations in BBB integrity and monocyte migration across endothelial cells.

### Acto-Myosin Belt

Additionally, there is thick band of actin cytoskeleton proximal to the apical junctional complex (AJC), which has been referred to as the perijunctional actomyosin ring (PAMR). This acto-myosin belt similarly serves a structural role, but when specifically activated it can serve as a physical disruption of paracellular barrier function—by squeezing/contracting an individual cell you pull it away from an adjacent one. Do this on a global level and gaps between cells will invariably form. The circumferential actomyosin belt regulates apical constriction. It is possible to modulate the acto-myosin belt with, for example, calcium chelators and Lulu proteins (Lulu1 and Lulu2).

### Agents for Transdermal Administration

A wide variety of therapeutic agents can be used in a transdermal delivery formulation, including anesthetics, fat removal compounds, nutrients, nonsteroidal anti-inflammatory drugs (NSAIDs) agents for the treatment of migraine, hair growth modulators, antifungal agents, anti-viral agents, vaccine components, tissue volume enhancing compounds, anti-cellulite therapeutics, wound healing compounds, compounds useful to effect smoking cessation, agents for prevention of collagen shrinkage, wrinkle relief compounds such as Botox®, skin-lightening compounds, compounds for relief of bruising, cannabinoids including cannabidiols for the treatment of epilepsy, compounds for adipolysis, compounds for the treatment of hyperhidrosis, acne therapeutics, pigments for skin coloration for medical or cosmetic tattooing, sunscreen compounds, hormones, insulin, corn/callous removers, wart removers, anesthetics, epinephrine and generally any therapeutic or prophylactic agent for which transdermal delivery is desired. As noted above, the delivery can simply effect transport across the skin into a localized subdermal location, such as treatment of nail fungus or modulation of hair growth or can effect systemic delivery such as is desirable in some instances where vaccines are used.

In any of the anesthetic compositions of a transdermal delivery formulation, it may be desirable to administer the epinephrine in tandem with a transdermal anesthetic. Alternatively, treatment of the epinephrine with a chelator, such as the iron chelator Desferal® may stabilize the epinephrine sufficiently to include it in the transdermal delivery formulation.

A transdermal delivery formulation can also comprise mixtures wherein the components interact synergistically and induce skin permeation enhancements better than that induced by the individual components. Synergies between chemicals can be exploited to design potent permeation enhancers that overcome the efficacy limitations of single enhancers. Several embodiments disclosed herein utilize one or more distinct permeation enhancers.

For topical administration, and in particular transdermal administration, a transdermal delivery formulation can comprise penetrants including either or both chemical penetrants (CPEs) and peptide-based cellular penetrating agents (CPPs) that encourage transmission across the dermis and/or across membranes including cell membranes, as would be the case in particular for administration by suppository or intranasal administration, but for transdermal administration as well. In some embodiments, suitable penetrants include those that are described in the above-referenced US2009/0053290 ('290), WO2014/209910 (910), and WO2017/127834. In addition to transdermal delivery formulations with penetrants, transdermal delivery can be effected by mechanically disrupting the surface of the skin to encourage penetration, or simply by supplying the formulation applied to the skin under an occlusive patch.

Alternatively, the transdermal delivery formulation can comprise a completion component as well as one or more electrolytes sufficient to impart viscosity and viscoelasticity, one or more surfactants and an alcohol. The completion component can be a polar liquid, a non-polar liquid or an amphiphilic substance. The penetrant can further comprise a keratinolytic agent effective to reduce thiol linkages, disrupt hydrogen bonding and/or effect keratin lysis and/or a cell penetrating peptide (sometimes referred to as a skin-penetrating peptide) and/or a permeation enhancer.

The transdermal delivery formulation can also include a gelling component. Suitable gelling components include isopropyl palmitate, ethyl laurate, ethyl myristate and isopropyl myristate. In some embodiments, a transdermal delivery formulation comprises a gelling agent in an amount less than 5% w/w of a transdermal delivery formulation. Certain hydrocarbons, such as cyclopentane, cyclooctane, trans-decalin, trans-pinane, n-pentane, n-hexane, n-hexadecane may also be used. In some embodiments, the transdermal delivery formulation comprises a mixture of xanthan gum, Sclerotium gum, pullulan, or a combination thereof in an amount less than 2% w/w, 5% w/w, or 10% w/w of the formulation. In some embodiments, a transdermal delivery formulation comprises Siligel™ in an amount between about 1-5% w/w or 5-15% w/w, or an equivalent mixture of xanthan gum, Sclerotium gum, and pullulan. In some embodiments, a transdermal delivery formulation comprises a mixture of caprylic triglycerides and capric triglycerides in amount less than 2% w/w, 8% w/w, or 10% w/w of the formulation. In some embodiments, a transdermal delivery formulation comprises Myritol® 312 in an amount between about 0.5-10% w/w, or an equivalent mixture of caprylic triglycerides and capric triglycerides.

In an embodiment, one or more anti-oxidants can be included, such as vitamin C, vitamin E, proanthocyanidin and a-lipoic acid typically in concentrations of 0.1%-2.5% w/w.

In some applications, in particular when a transdermal delivery formulation includes an anesthetic, epinephrine or an alternate vasoconstrictor, such as phenylephrine or epinephrine sulfate may be included in the formulation if a stabilizing agent is present. Otherwise, the epinephrine should be administered in tandem since epinephrine is not stable at alkali pH.

Another active agent is Withaferin A. Withaferin A inhibits tumor metastasis and manifests other anti-cancer activities, e.g., inhibition of the neovascularization associated with carcinoma, as well as cell proliferation. Withaferin A is also a leptin sensitizer with strong anti-diabetic properties that could induce healthy weight loss and beneficial effects on glucose metabolism.

Other agents include anti-metastatic agents including inhibitors of the src homology region 2-containing protein tyrosinase phosphatase (Shp2). A multiplicity of inhibitors of this activity is known, including Fumosorine, PHPS (NSC-87877) and NSC-117199, phenylhydrazonopyrazolone sulfonate (PHPS1), DCA, cryptotanshinone, 11-B08 and #220-324, metalloproteinases-2 and -9 (MMP-2 and MMP-9) and certain cathepsins, in particular B, D and L.

Other agents include inhibitors of E-cadherin and of epidermal growth factor receptor (EGFR). Known inhibitors include erlotinib, an anti-integrin drug (Cilengitide), Cariporide, Eniporide and Amiloride.

A transdermal delivery formulation can include other components that act as excipients or serve purposes other than for their therapeutic effects. For example, preservatives like antioxidants e.g., ascorbic acid or α-lipoic acid and antibacterial agents may be included. Other components apart from therapeutically active ingredients and components that are the primary effectors of dermal penetration may include those provided for aesthetic purposes such as menthol or other aromatics, and components that affect the physical state of the composition such as emulsifiers, for example, Durosoft® (which is a mixture of thermoplastic polyurethane and polycarbonate). Typically, these ingredients are present in very small percentages of the compositions. It is understood that these latter ancillary agents are neither therapeutically ingredients nor are they components that are primarily responsible for penetration of the skin. The components that primarily effect skin penetration have been detailed as described above. However, some of these substances have some capability for effecting skin penetration. See, for example, Kunta, J. R. et al, J. Pharm. Sci. (1997) 86:1369-1373, describing penetration properties of menthol.

For administration of anesthetics as the therapeutic agent, the local anesthetic can be one or more of the following: benzocaine, lidocaine, tetracaine, bupivacaine, cocaine, etidocaine, mepivacaine, pramoxine, prilocaine, procaine, chloroprocaine, oxyprocaine, proparacaine, ropivacaine, dyclonine, dibucaine, propoxycaine, chloroxylenol, cinchocaine, dexivacaine, diamocaine, hexylcaine, levobupivacaine, propoxycaine, pyrrocaine, risocaine, rodocaine, and pharmaceutically acceptable derivatives and bioisosteres thereof. Combinations of anesthetic agents may also be used. The anesthetic agent(s) are included in the composition in effective amount(s). Depending on the anesthetic(s) the amounts of anesthetic or combination is typically in the range of 1 w/w to 50% w/w. The compositions of the invention provide rapid, penetrating relief that is long lasting. The pain to be treated can be either traumatic pain and/or inflammatory pain.

In one embodiment, the anesthetic is administered to relieve the pain associated with invasive fat deposit removal. Specific removal of fat deposits has been attractive for both health and cosmetic reasons. Among the methods employed are liposuction and injection of a cytolytic agent for fat such as deoxycholic acid (DCA). For example, a series of patents issued or licensed to Kythera Biopharmaceuticals is directed to methods and compositions for non-surgical removal of localized fat that involves injecting compositions containing DCA or a salt thereof. Representative issued patents are directed to formulation (U.S. Pat. No. 8,367,649); method-of-use (U.S. Pat. Nos. 8,846,066; 7,622,130; 7,754,230; 8,298,556); and synthetic DCA (U.S. Pat. No. 7,902,387).

In this aspect of the invention, conventional invasive fat removal techniques are employed along with administering a pain-relieving effective agent—typically lidocaine or related anesthetics via transdermal administration. In some embodiments, the pain-relieving transdermal formulation is applied to the area experiencing pain immediately before, during or immediately after the invasive fat-removal procedure.

The compositions containing anesthetics are useful for temporary relief of pain and itching associated with minor burns, cuts, scrapes, skin irritations, inflammation and rashes due to soaps, detergents or cosmetics, or, as noted above, pain associated with removal of fat deposits.

In another embodiment, nutrients are supplied via transdermal administration. There are many occasions in which the formulations of the invention are useful. For athletes, a transdermal delivery formulation can deliver to tired muscles sufficient amounts of a neutralizing agent for lactic acid, such as ketone component, to relieve the burning sensation felt by the athlete due to the buildup of lactic acid. This permits the athlete to continue to perform at optimum level for longer periods of time. In addition, athletes or others “working out” are expending high amounts of energy and are in need of energy generation especially in those areas of their musculature that are involved in performing workouts and, therefore, need to consume large numbers of calories. These nutrients can be supplied directly rather than requiring oral ingestion which is counterproductive and relatively slow.

Emergency medical treatment of individuals requiring, for example, blood balancing agents including electrolytes and readily-metabolized nutrients, such as glucose, that would otherwise be administered intravenously can instead be non-invasively treated by massaging the formulation through the skin and thus permitting systemic delivery so that levels in the bloodstream are altered.

In addition to these applications, it has been noted that the administration of nutrients according to the invention also assuages feelings of hunger. Therefore, a transdermal delivery formulation of the invention and methods of the invention are useful in promoting weight loss as the caloric intake required to assuage feelings of hunger is lower than that ordinarily experienced by consuming food conventionally. Thus, in addition to individuals requiring extra calories or metabolic balancers because of exertion and in addition to those unable to feed themselves orally, suitable subjects for the methods of the invention include individuals seeking to control their caloric intake in order to adjust their weight. In view of the generally acknowledged obesity epidemic in the United States in particular, this is an important group of subjects benefitting from the methods of the invention.

It is clear that the nature of the desired ingredients will vary depending on the object of the administration. Simple nutrients such as amino acids, glucose, fructose, simple fats, various vitamins, cofactors and antioxidants as well as somewhat more complex foodstuffs can be administered as well as neutralizing agents, depending on the need.

In some embodiments, the components for athletic performance include beta-alanine, L-carnitine, adenosine triphosphate, dextrose, creatine monohydrate, beta hydroxy-betamethylbutyrate (HMB), branched chain amino acids (leucine, isoleucine, valine), glutathione, sodium phosphate, and caffeine. Components for medical nutrition include amino acids, dextrose, lipids, Na⁺, K⁺, Ca²⁺, Mg²⁺, acetate, Cl⁻, P, multivitamin, and trace elements. While components for weight loss include conjugated linoleic acids, ephedra, caffeine, and salicin.

Certain embodiments of a transdermal delivery formulation provided herein may be supplemented with formulation components described in greater detail in the inventor's related applications, including U.S. application Ser. No. 16/132,358 filed Sep. 14, 2018, entitled ‘Methods and Formulations For Transdermal Administration Of Buffering Agents’, International Patent Application No. PCT/US18/51250 filed Sep. 14, 2018, entitled ‘Methods of Administration and Treatment’, and International Patent Application PCT/US18/28017 by Bruce Sand filed Apr. 17, 2018, entitled ‘Parental non-systemic administration of buffering agents for inhibiting metastasis of solid tumors, hyperpigmentation and gout’, all incorporated by reference in their entirety herein.

In another aspect, certain embodiments are directed to a sustained release drug delivery platform releases a therapeutic compound or compounds disclosed and made as a formulation described herein over a period of about 3 days after administration, about 7 days after administration, about 10 days after administration, about 15 days after administration, about 20 days after administration, about 25 days after administration, about 30 days after administration, about 45 days after administration, about 60 days after administration, about 75 days after administration, or about 90 days after administration. In other aspects of this embodiment, a sustained release drug delivery platform releases a therapeutic compound or compounds disclosed herein with substantially first order release kinetics over a period of at least 3 days after administration, at least 7 days after administration, at least 10 days after administration, at least 15 days after administration, at least 20 days after administration, at least 25 days after administration, at least 30 days after administration, at least 45 days after administration, at least 60 days after administration, at least 75 days after administration, or at least 90 days after administration.

The formulation described in this specification may also comprise more than one therapeutic compound as desired for the particular indication being treated, preferably those with complementary activities that do not adversely affect the other proteins. A transdermal delivery formulation to be used for in vivo administration can be sterile. This can be accomplished, for example, by filtration through sterile filtration membranes, prior to, or following, preparation of a transdermal delivery formulation or other methods known in the art, including pasteurization.

Packaging and instruments for administration may be determined by a variety of considerations, such as the volume of material to be administered, the conditions for storage, whether skilled healthcare practitioners will administer or patient self-compliance, the dosage regime, the geopolitical environment (e.g., exposure to extreme conditions of temperature for developing nations), and other practical considerations.

In certain embodiments, kits can comprise one or more cream or lotion comprising one or more formulations described herein. In various embodiments, the kit can comprise formulation components for transdermal, topical, or subcutaneous administration, formulated to be administered as an emulsion coated patch. In all of these embodiments and others, the kits can contain one or more lotion, cream, patch, or the like in accordance with any of the foregoing, wherein each patch contains a single unit dose for administration to a subject.

Imaging components can optionally be included, and the packaging also can include written or web-accessible instructions for using a transdermal delivery formulation. A container can include, for example, a vial, bottle, patch, syringe, pre-filled syringe, tube or any of a variety of formats well known in the art for multi-dispenser packaging.

### Methods

The application method is determined by the nature of the treatment but may be less critical than the nature of the formulation itself. If the application is to a skin area, it may be helpful in some instances to prepare the skin by cleansing or exfoliation. In some instances, it is helpful to adjust the pH of the skin area prior to application of a transdermal delivery formulation itself. The application of a transdermal delivery formulation may be by simple massaging onto the skin or by use of devices such as syringes or pumps. Patches could also be used. In some cases, it is helpful to cover the area of application to prevent evaporation or loss of a transdermal delivery formulation.

Where the application area is essentially skin, it is helpful to seal-off the area of application subsequent to supplying a transdermal delivery formulation and allowing the penetration to occur so as to restore the skin barrier. A convenient way to do this is to apply a composition comprising linoleic acid which effectively closes the entrance pathways that were provided by the penetrants of the invention. This application, too, is done by straightforward smearing onto the skin area or can be applied more precisely in measured amounts.

In addition to the compositions and formulations of the invention per se, the methods may employ a subsequent treatment with linoleic acid. As transdermal treatments generally open up the skin barrier, which is, indeed, their purpose, it is useful to seal the area of application after the treatment is finished. Thus, treatment with a transdermal delivery formulation may be followed by treating the skin area with a composition comprising linoleic acid to seal off the area of application. The application of linoleic acid is applicable to any transdermal procedure that results in impairing the ability of the skin to act as a protective layer. Indeed, most transdermal treatments have this effect as their function is to allow the active component to pass through the epidermis to the dermis at least, and, if systemic administration is achieved, through the dermis itself.

Methods for treating, preventing or ameliorating a disease, disorder, a condition, or a symptom thereof or a condition related thereto are provided herein using a transdermal delivery formulation for transdermal delivery described herein below. The methods provided herein may comprise or consist of topically administering one or more of a transdermal delivery formulation described herein to skin of a subject in need thereof. Preferred, but non-limiting embodiments are directed to methods for treating, preventing, inhibiting or ameliorating a disease, disorder, a condition, or a symptom described below.

Formulations provided herein are also used in methods of treating a cancer or tumor, including but not limited to Adrenocortical Carcinoma, Basal Cell Carcinoma, Bladder Cancer, Bone Cancer, Brain Tumor, Breast Cancer, Cervical Cancer, Colon Cancer, Colorectal Cancer, Esophageal Cancer, Retinoblastoma, Gastric (Stomach) Cancer, Gastrointestinal Tumors, Glioma, Head and Neck Cancer, Hepatocellular (Liver) Cancer, Islet Cell Tumors (Endocrine Pancreas), Kidney (Renal Cell) Cancer, Laryngeal Cancer, Non-small Cell Lung Cancer, Small Cell Lung Cancer, Medulloblastoma, Melanoma, Pancreatic Cancer, Prostate Cancer, Renal Cancer, Rectal cancer, and Thyroid Cancer.

### Urinary and Renal Stones and Related Disorders

Kidney stones (renal lithiasis, nephrolithiasis) are common in humans and animals, and they typically comprise hard deposits made of minerals and salts that form inside the bladder, kidneys, and urinary tract. Such stones often form when the urine becomes concentrated, allowing minerals to crystallize and stick together. Also, when a subject does not drink sufficient water there can be an accumulation of uric acid that is believed to be correlated with the formation of such stones. An excessively acidic environment in the urine of a subject is also thought to lead to the formation of kidney stones. They can be quite painful and can lead to complications such as the blocking of the tube connecting the kidney to the bladder. Embodiments of a transdermal delivery formulation provided herein have been found to be useful for the treatment, inhibition, amelioration of urinary and renal stones in a subject.

Accordingly, other embodiments provided herein are directed to methods of urinary and renal stones and related disorders. In an exemplary embodiment, a method of ameliorating or treating a urinary stone in accordance with the invention typically comprises topically and/or transdermally administering an effective amount of a transdermal delivery formulation to a patient having a urinary stone and in need thereof, wherein said administration is effective to ameliorate, treat or reduce the symptoms of the urinary stone in said patient.

Examples of such conditions involving stones include, but not limited to bladder stones, kidney stones (calcium, calcium oxalate, calcium phosphate, cystine, magnesium ammonium phosphate, uric acid, struvite), renal stones, bilateral stone disease, urolithiasis during pregnancy, pediatric stones, stones in animals (e.g. urinary stones in animals), stones in patients with solitary kidneys, nephrolithiasis, other types of stones (e.g. bladder, urinary), patients with bleeding diathesis and related disorders, urolithiasis, as well as in conjunction with medical or surgical procedures such as a lithotripsy or ureteroscopy.

### Skin Disorders

Other embodiments are directed to methods of treating a skin condition or disorder in a patient. These embodiments typically comprise topically and/or transdermally administering an effective amount of a transdermal delivery formulation to a patient having a skin condition or disorder and in need thereof, wherein said administration is effective to ameliorate, treat or reduce the symptoms of the skin condition or disorder.

An exemplary skin disorder that is treated herein in particular embodiments is melasma. Melasma is a common skin problem that leads to skin pigmentation problems such as brown to gray-brown patches, usually on the face, cheeks, bridge of their nose, forehead, chin, and above their upper lip.

Melasma is believed to be triggered or worsened by birth control pills, pregnancy, and hormone therapy, stress, thyroid disease, and sun exposure. Sun exposure is believed to cause melasma because ultraviolet rays affect the cells that control pigment (melanocytes).

Thus, in certain embodiments methods of treating melasma are provided that comprise topically and/or transdermally administering an effective amount of a transdermal delivery formulation to a patient having melasma and in need thereof, wherein said administration is effective to ameliorate, treat or reduce the symptoms of the melasma. In some embodiments, methods of the invention use a transdermal delivery formulation provided herein in conjunction with or co-administered with another treatment for melasma (e.g. sun protection or a sun screen).

Another disorder or condition of the skin that is treated is skin damage. These embodiments typically comprise topically and/or transdermally administering an effective amount of a formulation to a patient having skin damage and in need thereof, wherein said administration is effective to ameliorate, treat or reduce the skin damage or symptoms associated with the skin damage.

Other embodiments are directed to rejuvenating skin, and accordingly methods of rejuvenating skin are provided that comprise topically and/or transdermally administering an effective amount of a transdermal delivery formulation to a subject in need of skin rejuvenation.

In certain embodiments, methods are provided that prevent or ameliorate collagen acylation in the skin of a patient. Alternative embodiments are also directed to the pre-treatment of skin to prevent or ameliorate skin damage caused by collagen acylation and other factors.

### Administration and Dosing

A transdermal delivery formulation provided herein can be topically administered in any form. For administration for the treatment of skin conditions a sufficient amount of the topical composition can be applied onto a desired area and surrounding skin, for example, in an amount sufficient to cover a desired skin surface. A transdermal delivery formulation can be applied to any skin surface, including for example, facial skin, and the skin of the hands, neck, chest and/or scalp.

In applying a transdermal delivery formulation of the invention, a transdermal delivery formulation itself is simply placed on the skin and spread across the surface and/or massaged to aid in penetration. The amount of transdermal delivery formulation used is typically sufficient to cover a desired surface area. In some embodiments, a protective cover is placed over the formulation once it is applied and left in place for a suitable amount of time, i.e., 5 minutes, 10 minutes, 20 minutes or more; in some embodiments an hour or two. The protective cover can simply be a bandage including a bandage supplied with a cover that is impermeable to moisture. This essentially locks in the contact of a transdermal delivery formulation to the skin and prevents distortion of a transdermal delivery formulation by evaporation in some cases. The composition may be applied to the skin using standard procedures for application such as a brush, a syringe, a gauze pad, a dropper, or any convenient applicator. More complex application methods, including the use of delivery devices, may also be used, but are not required. In an alternative to administering topically to intact skin, the surface of the skin may also be disrupted mechanically by the use of spring systems, laser powered systems, systems propelled by Lorentz force or by gas or shock waves including ultrasound and may employ microdermabrasion such as by the use of sandpaper or its equivalent or using microneedles or electroporation devices. Simple solutions of the agent(s) as well as the above-listed formulations that penetrate intact skin may be applied using occlusive patches, such as those in the form micro-patches. External reservoirs of the formulations for extended administration may also be employed.

Accordingly, in certain embodiments alternative methods of administering therapeutic compounds, agents, drugs through intact skin are provided. As nonlimiting examples, these alternative methods might be selected from the following lists: on basis of working mechanism, spring systems, laser powered, energy-propelled, Lorentz force, gas/air propelled, shock wave (including ultrasound), on basis of type of load, liquid, powder, projectile, on basis of drug delivery mechanism, nano-patches, sandpaper (microdermabrasion), iontophoresis enabled, microneedles, on basis of site of delivery, intradermal, intramuscular, and subcutaneous injection. Other suitable delivery mechanisms include microneedle drug delivery, such as 3M Systems, Glide SDI (pushes drug as opposed to “firing” drug), MIT low pressure injectors, micropatches (single use particle insertion device), microelectro mechanical systems (MEMS), dermoelectroporation devices (DEP), transderm ionto system (DEP), TTS transdermal therapeutic systems, membrane-moderated systems (drug reservoir totally encapsulated in a shallow compartment), adhesive diffusion-controlled system (drug reservoir in a compartment fabricated from drug-impermable metallic plastic backing), matrix dispersion type system (drug reservoir formed by homogeneously dispersing drug solids in a hydrophilic or lipophilic polymer matrix molder into medicated disc), and microreservoir system (combination of reservoir and matrix dispersion-type drug delivery system).

It has been found, generally, that the requirements for effective penetration of the skin in the case of buffers as active agents are less restrictive than those required for alternative agents useful in preventing cancer metastasis. In addition, although for these indications' delivery to the locus of the solid tumor, including melanoma, or melasma or gout is desirable, effective systemic pH alteration can be used as a way to diagnose the effectiveness of penetration when topical administration is employed.

The application method is determined by the nature of the treatment but may be less critical than the nature of a transdermal delivery formulation itself. If the application is to a skin area, it may be helpful in some instances to prepare the skin by cleansing or exfoliation. In some instances, it is helpful to adjust the pH of the skin area prior to application of the formulation itself. The application of a transdermal delivery formulation may be by simply massaging onto the skin or by use of devices such as syringes or pumps. Patches could also be used. In some cases, it is helpful to cover the area of application to prevent evaporation or loss of a transdermal delivery formulation.

Where the application area is essentially skin, it is helpful to seal-off the area of application subsequent to supplying a transdermal delivery formulation and allowing the penetration to occur so as to restore the skin barrier. A convenient way to do this is to apply a composition comprising linoleic acid which effectively closes the entrance pathways that were provided by the penetrants of the invention. This application, too, is done by straightforward smearing onto the skin area or can be applied more precisely in measured amounts.

In some embodiments, the disclosure is directed to administering a therapeutic agent in combination with a formulation or method provided herein. A wide variety of therapeutic agents may be used in a transdermal delivery formulation or compositions and formulations for other routes of administration, including anesthetics, fat removal compounds, nutrients, nonsteroidal anti-inflammatory drugs (NSAIDs) agents for the treatment of migraine, hair growth modulators, antifungal agents, anti-viral agents, vaccine components, tissue volume enhancing compounds, anti-cellulite therapeutics, wound healing compounds, compounds useful to effect smoking cessation, agents for prevention of collagen shrinkage, wrinkle relief compounds such as Botox®, skin-lightening compounds, compounds for relief of bruising, cannabinoids including cannabidiols for the treatment of epilepsy, compounds for adipolysis, compounds for the treatment of hyperhidrosis, acne therapeutics, pigments for skin coloration for medical or cosmetic tattooing, sunscreen compounds, hormones, insulin, corn/callous removers, wart removers, and generally any therapeutic or prophylactic agent for which transdermal delivery is desired. As noted above, the delivery may simply affect transport across the skin into a localized subdermal location, such as treatment of nail fungus or modulation of hair growth or may affect systemic delivery such as is desirable in some instances where vaccines are used.

In addition to a transdermal delivery formulation of the invention per se, the methods may employ a subsequent treatment with linoleic acid. As transdermal treatments generally open up the skin barrier, which is, indeed, their purpose, it is useful to seal the area of application after the treatment is finished. Thus, treatment with a transdermal delivery formulation may be followed by treating the skin area with a composition comprising linoleic acid to seal off the area of application. The application of linoleic acid is applicable to any transdermal procedure that results in impairing the ability of the skin to act as a protective layer. Indeed, most transdermal treatments have this effect as their function is to allow active ingredients to pass through the epidermis to the dermis at least, and, if systemic administration is achieved, through the dermis itself.

Additional therapeutic agents may be included in the compositions. For example, hydrocortisone or hydrocortisone acetate may be included in an amount ranging from 0.25% w/w to about 0.5% w/w. Menthol, phenol, and terpenoids, e.g., camphor, can be incorporated for cooling pain relief. For example, menthol may be included in an amount ranging from about 0.1% w/w to about 1.0% w/w.

A transdermal delivery formulation can be applied in a single, one-time application, once a week, once a bi-week, once a month, or from one to twelve times daily, for a period of time sufficient to alleviate a condition, disease, disorder, symptoms, for example, for a period of time of one week, from 1 to 12 weeks or more, from 1 to 6 weeks, from 2 to 12 weeks, from 2 to 12 weeks, from 2 to 8 weeks, from 2 to 6 weeks, from 2 to 4 weeks, from 4 to 12 weeks, from 4 to 8 weeks, or from 4 to 6 weeks. The present compositions can be administered, for example, at a frequency of once per day to hourly if needed. The presently described formulations can be topically administered once or more per day for a period of time from 1 week to 4 weeks, of from 1 week to 2 weeks, for 1 week, for 2 weeks, for 3 weeks, for 4 weeks, or for 4 weeks or more. In some instances, it may also be desirable to continue treatment indefinitely for example to inhibit or prevent carcinogenesis or for improving, extending the duration of remission, or maintaining remission of a cancer or another disease or disorder. A suitable administration for a transdermal delivery formulation comprising a skin cream, lotion or ointment, for example is once, twice, three, four times daily, or hourly if needed.

As described above, if desired, other therapeutic agents can be employed in conjunction with those provided in the above-described compositions. The amount of active ingredients that may be combined with the carrier materials to produce a single dosage form will vary depending upon the host treated, the nature of the disease, disorder, or condition, and the nature of the active ingredients.

It is understood that a specific dose level for any particular patient will vary depending upon a variety of factors, including the activity of the specific active agent; the age, body weight, general health, sex and diet of the patient; the time of administration; the rate of excretion; possible drug combinations; the severity of the particular condition being treated; the area to be treated and the form of administration. One of ordinary skill in the art would appreciate the variability of such factors and would be able to establish specific dose levels using no more than routine experimentation.

Pharmacokinetic parameters such as bioavailability, absorption rate constant, apparent volume of distribution, unbound fraction, total clearance, fraction excreted unchanged, first-pass metabolism, elimination rate constant, half-life, and mean residence time can be determined by methods well known in the art.

A transdermal delivery formulation in accordance with the subject matter described herein may be a topical dosage form packaged in, for example, a multi-use or single-use package, including for example, a tube, a bottle, a pump, a container or bottle, a vial, a jar, a packet, or a blister package.

Single dosage kits and packages containing a once per day amount of the transdermal delivery formulation may be prepared. Single dose, unit dose, and once-daily disposable containers of the transdermal delivery formulation are also provided.

The present transdermal delivery formulation remains stable in storage for periods including up to about 5 years, between about 3 months and about 5 years, between about 3 months and about 4 years, between about 3 months and about 3 years, and alternately any time period between about 6 months and about 3 years.

If desired, other therapeutic agents can be employed in conjunction with those provided in the above-described compositions. The amount of active ingredients that may be combined with the carrier materials to produce a single dosage form will vary depending upon the host treated, the nature of the disease, disorder, or condition, and the nature of the active ingredients

It is understood that a specific dose level for any particular patient will vary depending upon a variety of factors, including the activity of the specific active agent; the age, body weight, general health, sex and diet of the patient; the time of administration; the rate of excretion; possible drug combinations; the severity of the particular condition being treated; the area to be treated and the form of administration. One of ordinary skill in the art would appreciate the variability of such factors and would be able to establish specific dose levels using no more than routine experimentation.

Pharmacokinetic parameters such as bioavailability, absorption rate constant, apparent volume of distribution, unbound fraction, total clearance, fraction excreted unchanged, first-pass metabolism, elimination rate constant, half-life, and mean residence time can be determined by methods well known in the art.

A transdermal delivery formulation in accordance with the subject matter described herein may be a topical dosage form packaged in, for example, a multi-use or single-use package, including for example, a tube, a bottle, a pump, a container or bottle, a vial, a jar, a packet, or a blister package.

Single dosage kits and packages containing a once per day amount of the transdermal delivery formulation may be prepared. Single dose, unit dose, and once-daily disposable containers of the transdermal delivery formulation are also provided.

Dosing can be single dosage or cumulative (serial dosing), and can be readily determined by one skilled in the art. A transdermal delivery formulation of the present invention may be administered once, twice, three, four, five, six, seven, eight, nine, ten, eleven, twelve, thirteen, fourteen, fifteen, sixteen, seventeen, eighteen, nineteen, twenty or more times to a subject. For instance, treatment of a disease may comprise a one-time administration of an effective dose of a transdermal delivery formulation as disclosed herein. Alternatively, treatment of a disease may comprise multiple administrations of an effective dose of a transdermal delivery formulation as carried out over a range of time periods, such as, e.g., once daily, twice daily, trice daily, once every few days, or once weekly. The timing of administration can vary from individual to individual, depending upon such factors as the severity of an individual's symptoms. For example, an effective dose of a transdermal delivery formulation as disclosed herein can be administered to an individual once daily for an indefinite period of time, or until the individual no longer requires therapy. A person of ordinary skill in the art will recognize that the condition of the individual can be monitored throughout the course of treatment and that the effective amount of a transdermal delivery formulation disclosed herein that is administered can be adjusted accordingly. In one embodiment, a transdermal delivery formulation as disclosed herein is capable of decreasing the time to resolve the symptoms of a disease, including in an individual suffering from a disease by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment.

A transdermal delivery formulation disclosed herein may comprise an anti-cancer transdermal delivery formulation in a therapeutically effective amount. As used herein, the term “effective amount” is synonymous with “therapeutically effective amount”, “effective dose”, or “therapeutically effective dose” and when used in reference to reducing or maintaining a cancer cell population and/or tumor cell size in an individual refers to the minimum dose of a cancer therapeutic disclosed herein necessary to achieve the desired therapeutic effect and includes a dose sufficient to reduce or maintain of cancer cell population and/or tumor cell size in an individual. The effectiveness of an anti-cancer transdermal delivery formulation disclosed herein capable of reducing or maintaining a cancer cell population and/or tumor cell size in an individual can be determined by observing an improvement in an individual based upon one or more clinical symptoms, and/or physiological indicators associated with reducing or maintaining a cancer cell population and/or tumor cell size in an individual. Maintenance or a reduction of cancer cell population and/or tumor cell size can be indicated by a reduced need for a concurrent therapy. The effectiveness of an anti-cancer transdermal delivery formulation disclosed herein capable of reducing or maintaining a cancer cell population and/or tumor cell size in an individual can be determined by observing an improvement in an individual based upon one or more clinical symptoms, and/or physiological indicators associated with a reduction or maintenance of cancer cell population and/or tumor cell size. The effectiveness of an anti-cancer transdermal delivery formulation disclosed herein is also capable of prolonging the life of an individual as compared to the same individual if the anti-cancer transdermal delivery formulation is not administered. The effectiveness of anti-cancer transdermal delivery formulation disclosed herein is also capable of enhancing the quality of life of an individual as compared to the same individual if the anti-cancer transdermal delivery formulation is not administered.

The appropriate effective amount of an anti-cancer transdermal delivery formulation disclosed herein to be administered to reduce or maintain of a cancer cell population and/or tumor cell size in an individual condition can be determined by a person of ordinary skill in the art by taking into account factors, including the measured number of cancer cells in blood samples or biopsies or CAT scans, PET scans, NMR and/or sonograms taken from or of the individual, the particular characteristics, history and risk factors of the patient, such as, e.g., age, weight, general health and the like, or any combination thereof. Additionally, where repeated administration of an anti-cancer transdermal delivery formulation is used, an effective amount of an anti-cancer transdermal delivery formulation will further depend upon factors, including the frequency of administration, the half-life of the anti-cancer transdermal delivery formulation, or any combination thereof. It is known by a person of ordinary skill in the art that an effective amount of an anti-cancer transdermal delivery formulation disclosed herein can be extrapolated from in vitro assays and in vivo administration studies using animal models prior to administration to humans or animals.

Wide variations in the necessary effective amount are to be expected in view of the differing efficiencies of the various routes of administration. For instance, oral administration of an anti-cancer transdermal delivery formulation disclosed herein generally would be expected to require higher dosage levels than administration by inhalation. Similarly, systemic administration of an anti-cancer transdermal delivery formulation disclosed herein would be expected to require higher dosage levels than a local administration. Variations in these dosage levels can be adjusted using standard empirical routines of optimization, which are well-known to a person of ordinary skill in the art. The precise therapeutically effective dosage levels and patterns are preferably determined by the attending physician in consideration of the above-identified factors. One skilled in the art will recognize that the condition of the individual can be monitored throughout the course of therapy and that the effective amount of a cancer therapeutic disclosed herein that is administered can be adjusted accordingly.

Aspects of the present specification disclose, in part, reduction or maintenance of cancer cell population and/or tumor cell size in an individual. As used herein, the term “treating,” refers to reduction or maintenance of cancer cell population and/or tumor cell size in an individual. For example, the term “treating” can mean reduction or maintenance of cancer cell population and/or tumor cell size levels in an individual by, e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 100%. The actual symptoms associated with cancer, including the detection of cancer cell population and/or tumor cell size are well known and can be determined by a person of ordinary skill in the art by using commonly known testing means, including blood tests, CT scans sonograms and other tests known to those of ordinary skill. Those of skill in the art will know the appropriate symptoms or indicators associated with cancer and will know how to determine if an individual is a candidate for treatment as disclosed herein.

In an embodiment, a first anti-cancer transdermal delivery formulation is administered to an individual and at a later date, a second anti-cancer transdermal delivery formulation is administered to the same individual. In an embodiment, a first anti-cancer transdermal delivery formulation is administered to an individual at the same time as a second anti-cancer transdermal delivery formulation is administered to the individual.

In some embodiments, a cannabinoid is provided to a subject via transdermal administration. The cannabinoid can be a crystalline cannabidiol.

### Example 1

Murine Urine pH Study

In this experiment, the ability of a buffer to be absorbed, in this example, sodium carbonate, was examined in mice in formulations that either included or did not include epithelial junction modifiers. The mice were administered two transdermal formulations. One transdermal formulation, hereinafter referred to as Formulation A, included epithelial junction modifiers. The included epithelial junction modifiers included in Formulation A were—1% EDTA, 0.5% Triethanolamine, and 1% Sodium Decanoate The other formulation, hereinafter referred to as Formulation B did not include any epithelial junction modifiers. Both Formulations A and B did include a buffer. Namely, both Formulation A and B contained 7.5% sodium carbonate.

Formulations A and B were applied topically to mice at a dose of 300 μl/day total of the buffer sodium carbonate. The doses were applied three times a day at a dose of 100 μl of the buffer sodium carbonate. Formulations A and B were applied to the mice for three days (nine total applications).

Urine samples were collected at various time points during the three days the Formulations were applied and for a short period of time thereafter. The urine samples were tested to determine their pH. The higher the pH, the more buffer in Formulation A or Formulation B that was able to cross the skin and enter the blood stream.

The results of the experiment are presented in Table 1.

TABLE 1

| | Average | Average | Average | Average |
|---|---|---|---|---|
| | Pre-Dose | Post-Dose | Change | % |
| Formulation | pH | pH | in pH | change |
| A | 5.61 | 6.27 | 0.66 | 11.82% |
| B (with Epithelial | 5.68 | 7.63 | 1.94 | 34.21% |
| Junction Modifiers) | | | | |

Based on the results set forth in Table 1, Formulation A that contained epithelial junction modifiers was able to raise the pH of the urine of mice that received a topical administration of Formulation A by significantly more (34.21% or about 3× more) than Formulation B (11.82%). The results support the ability of the epithelial junction modifiers to increase transdermal penetration of the skin following topical administration of Formulation A versus Formulation B.

In closing, regarding the exemplary embodiments of the present invention as shown and described herein, it will be appreciated that the formulations disclosed herein are configured for buffering therapy with or without an additional therapeutic agent. Because the principles of the invention may be practiced in a number of configurations beyond those shown and described, it is to be understood that the invention is not in any way limited by the exemplary embodiments but is generally directed to a transdermal formulation and is able to take numerous forms to do so without departing from the spirit and scope of the invention. It will also be appreciated by those skilled in the art that the present invention is not limited to the particular components disclosed but may instead entail other functionally comparable formulation components, now known or later developed, without departing from the spirit and scope of the invention.

Certain embodiments of the present invention are described herein, including the best mode known to the inventor(s) for carrying out the invention. Of course, variations on these described embodiments will become apparent to those of ordinary skill in the art upon reading the foregoing description. The inventor(s) expect skilled artisans to employ such variations as appropriate, and the inventor(s) intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein. Similarly, as used herein, unless indicated to the contrary, the term “substantially” is a term of degree intended to indicate an approximation of the characteristic, item, quantity, parameter, property, or term so qualified, encompassing a range that can be understood and construed by those of ordinary skill in the art.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc. —for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (along with equivalent open-ended transitional phrases thereof such as “including,” “containing” and “having”) encompasses all the expressly recited elements, limitations, steps and/or features alone or in combination with un-recited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amendment for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim, whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (along with equivalent open-ended transitional phrases thereof) includes within its meaning, as a limiting case, claimed subject matter specified by the closed-ended transitional phrases “consisting of” or “consisting essentially of.” As such, embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

While aspects of the invention have been described with reference to at least one exemplary embodiment, it is to be clearly understood by those skilled in the art that the invention is not limited thereto. Rather, the scope of the invention is to be interpreted only in conjunction with the appended claims and it is made clear, here, that the inventor(s) believe that the claimed subject matter is the invention.

1. A transdermal delivery formulation, wherein the transdermal delivery formulation comprises one or more junctional protein modulators and/or comprises one or more acto-myosin belt modulators.

2. The transdermal delivery formulation of claim 1, wherein the formulation includes an active agent to treat a disease or promote health and well-being, the formulation includes an anesthetic, and/or the formulation includes a cannabinoid.

3. The transdermal delivery formulation of claim 2, wherein the disease is a cancer, a kidney disease, gout, melasma, a heart condition, or a dermal disease.

4.-5. (canceled)

6. The transdermal delivery formulation of claim 1, wherein the one or more junctional protein modulators is at least one of Clostridium perfringens enterotoxin, ZOT, AT1002, chitosan, a calcium chelator, sodium caprate, FDFWITP, PN159, 1-1-palmitoyl-2-glutaroyl-sn-glycero-3-phosphocholine (PGPC), EDTA, oleic acid, a bile acid, sphingolipid sphingosine-1-phosphate (S1P), dihydro-S1P, a prostanoid, a leukotriene, arachidonic acid, eicosapentaenoic acid, an ergot alkaloid, calyculin-A, okadaic acid, anticholinesterase drugs or histamine; and wherein the one or more acto-myosin belt modulators is a calcium chelator.

7.-12. (canceled)

13. A method of transdermal delivery of an active agent, the method comprising steps of:
a) applying a transdermal delivery formulation to skin, nail, or hair follicle of a subject,
b) penetrating the stratum corneum,
c) modulating one or more junctional proteins, and
d) modulating acto-myosin belts.

14. The method of transdermal delivery of claim 13, wherein a junctional protein modulator is used in the step of modulating one or more junctional proteins, said junctional protein modulator selected from the group of Clostridium perfringens enterotoxin, ZOT, AT1002, chitosan, a calcium chelator, sodium caprate, FDFWITP, PN159, 1-1-palmitoyl-2-glutaroyl-sn-glycero-3-phosphocholine (PGPC), EDTA, oleic acid, a bile acid, sphingolipid sphingosine-1-phosphate (S1P), dihydro-S1P, a prostanoid, a leukotriene, arachidonic acid, eicosapentaenoic acid, an ergot alkaloid, calyculin-A, okadaic acid, anticholinesterase drugs or histamine.

15. The method of transdermal delivery of claim 13, wherein a calcium chelator is used in the step of modulating acto-myosin belts.

16. The method of transdermal delivery of claim 13, wherein the transdermal delivery formulation includes an active agent to treat a disease.

17. The method of transdermal delivery of claim 16, wherein the disease is a cancer, a kidney disease, gout, melasma, a heart condition or a dermal disease.

18. The method of transdermal delivery of claim 13, wherein the formulation includes an anesthetic and/or the formulation includes a cannabinoid.

19.-22. (canceled)

23. A method of enhancing absorption of an agent across epithelial cells of the intestine or of enhancing absorption of an agent through the blood brain barrier, the method comprising a step of modulating one or more junctional proteins.

24. The method of claim 23, wherein the step of modulating one or more junctional proteins uses a protein modulator selected from the group of Clostridium perfringens enterotoxin, ZOT, AT1002, chitosan, a calcium chelator, sodium caprate, FDFWITP, PN159, 1-1-palmitoyl-2-glutaroyl-sn-glycero-3-phosphocholine (PGPC), EDTA, oleic acid, a bile acid, sphingolipid sphingosine-1-phosphate (S1P), dihydro-S1P, a prostanoid, a leukotriene, arachidonic acid, eicosapentaenoic acid, an ergot alkaloid, calyculin-A, okadaic acid, anticholinesterase drugs or histamine.

25. The method of claim 23, wherein the step of modulating one or more junctional proteins uses a calcium chelator.

26.-28. (canceled)
